(19) United States
(12) Patent Application Publication
(10) Pub. No.: US 2003/0152652 A1
(43) Pub. Date: Aug. 14, 2003

(54) Use of echinacea as a hematinic agent

(76) Inventors: John D. Baker, Ontario (CA); Wendy P. O'Neill, Ontario (CA); Andrew Clarke, Victoria (AU)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/275,188

(22) PCT Filed: May 4, 2001
(86) PCT No.: PCT/CA01/00612

(51) Int. Cl. A61K 35/78

(52) U.S. Cl. 424/737

(57) ABSTRACT

A method, wherein a composition comprising Echinacea and a pharmaceutically acceptable carrier is administered to an animal or a human, in an amount effective to cause a hematinic effect in the animal or the human. The Echinacea composition of the present invention has minimal or no side effects and is effective, simple to prepare and relatively inexpensive.

### TECHNICAL FIELD

The present invention relates to the use of the dietary supplement Echinacea as a hematinic agent.

### BACKGROUND

Echinacea (coneflower) is a member of the family Asteracease. The genus Echinacea contains 9 currently accepted species, 3 of which thus far, Echinacea purpurea, Echinacea pallida and Echinacea augustifolia have been used as medicinals.

Active components of Echinacea include, but are not limited to, alkaloids, cichoric acid derivatives, complex polysaccharides, fatty acids, glycoproteins, glycosides, flavonoids, monoterpenes, caffeic acid derivatives, N-alkanes, alkylamides, isobutylamides, cynarin, polyacetylenes, echinolone and echinacoside. Because the chemistry of Echinacea is so complicated, the Echinacea component known as “echinacoside” is used as a marker to standardize both the maturity of the Echinacea plants and the potency of the extracts prepared from the Echinacea plants.

Historically, Echinacea has been widely used as an immunostimulator. It is reported to increase T-cell counts, improve killer cell activity, improve phagocytosis, stimulate release of interferon, stimulate production of alpha-, beta- and gamma globulins, inhibit the activity of hyaluronidase, stimulate proliferation of fibroblasts and accelerate wound healing. Further, Echinacea is reported to have antiviral activity, antibacterial activity, antifungal activity, antiprotozoal activity, antiinflammatory activity and antitumor activity.

Hematological disorders include, but are not limited to, anemias. Anemia is a manifestation of nutritional deficiency or disease. The major causes of anemia are iron deficiency, impaired red cell production, impaired red cell maturation, impaired red cell release from the marrow, acute blood loss and acute red cell destruction. Tests used to diagnose anemia include, but are not limited to, red blood cell count, hemoglobin, hematocrit, mean corpuscular volume, mean corpuscular hematocrit, mean corpuscular hemoglobin concentration and blood smear examination. Anemias, because they decrease the oxygen carrying capacity of the blood, result in a decrease in the performance parameters of an animal. Methods used to treat anemias include, but are not limited to, blood transfusions, corticosteroids, splenectomy, drug therapy and bone marrow transplantation. These methods are expensive, invasive and often have adverse side effects.

Even in the absence of a hematological disorder, an increase in performance parameters due to increased oxygen transport capacity of the blood including, but not limited to, increased stamina, increased endurance, increased agility, increased mental acuity, increased mental alertness and decreased stress without adverse side affects is clearly advantageous.

Therefore, there is the need for a dietary supplement for use in an animal, including a human, that can prevent hematological disorders, than can treat hematological disorders, that can stimulate erythropoiesis, that can enhance performance parameters, that has minimal side effects, and that is effective, simple to prepare and relatively inexpensive.

### SUMMARY OF THE INVENTION

The present invention provides a method, wherein a composition comprising Echinacea and a pharmaceutically acceptable carrier, hereinafter called an Echinacea composition, is administered to an animal in an amount effective to cause a hematinic effect in the animal. The Echinacea composition of the present invention has minimal side effects and is effective, simple to prepare and relatively inexpensive. The Echinacea composition of the present invention may be administered to humans and animals. It is to be understood that animals include domestic pets, farm animals, game animals, zoo animals, animals used for performance, and any other animals that may benefit from a hematinic effect. Animals include, but are not limited to, horses, dogs, cattle, swine, elk, deer, donkeys, mules, llamas, camels, elephants, chickens, ducks, cats, and the like.

The surprising ability of Echinacea to prevent hematological disorders, to treat hematological disorders, to stimulate erythropoiesis and to enhance performance parameters, while itself having minimal side-effects, addresses a long-felt unfulfilled need in the medical arts and provides an important benefit for animals, including humans.

Accordingly, it is an object of the present invention to provide a method for preventing hematological disorders in an animal, including a human.

Yet another object of the present invention is to provide a method for treating hematological disorders in an animal, including a human.

Still another object of the present invention is to provide a method for stimulating erythropoiesis in an animal, including a human.

Yet another object of the present invention is to provide a method for increasing the number of red blood cells in an animal, including a human.

It is another object of the present invention to provide a method for increasing the hemoglobin concentration in an animal, including a human.

It is still another object of the present invention to provide a method for enhancing performance parameters that depend on the oxygen carrying capacity of the blood in an animal, incuding a human.

Another object of the present invention is to provide a method for enhancing the stamina of an animal, including a human.

Yet another object of the present invention is to provide a method for enhancing the endurance of an animal, including a human.

It is still another object of the present invention to provide a method for enhancing the mental acuity of an animal, including a human.

It is another object of the present invention to provide a method for enhancing the mental alertness of an animal, including a human.

It is still another object of the present invention to provide a method for reducing the stress of an animal, including a human.

It is yet another object of the present invention to provide an Echinacea composition that has minimal side effects.

It is another object of the present invention to provide an Echinacea composition that is simple to prepare.

Yet another object of the present invention is to provide an Echinacea composition that is effective.

It is still another object of the present invention to provide an Echinacea composition that is relatively inexpensive.

These and other objects, features and advantages of the present invention will become apparent after review of the following detailed description of the disclosed embodiment ant the appended claims.

### BRIEF DESCRIPTION OF THE FIGURES

FIG. 1. Comparison of red blood cell counts and changes in red blood cell counts during 42 days of dietary supplementation with placebo syrup and with Echinacea syrup. Results are the mean±SED (Standard Error of Difference) for 8 horses per group.

FIG. 2. Comparison of hemoglobin concentrations and changes in hemoglobin concentrations during 42 days of dietary supplementation with placebo syrup and with Echinacea syrup. Results are the mean±SED for 8 horses per group.

### DETAILED DESCRIPTION OF THE INVENTION

The present invention provides a method, wherein a composition comprising Echinacea and a pharmaceutically acceptable carrier, hereinafter called an Echinacea composition, is administered to an animal in an amount effective to cause a hematinic effect in the animal. The Echinacea composition of the present invention has minimal side effects and is effective, simple to prepare and relatively inexpensive.

As defined herein, “hematinic” includes improving the condition of the blood by increasing the oxygen carrying capacity of the blood.

As defined herein “hematinic effect” includes preventing a hematological disorder, treating a hematological disorder, stimulating erythropoiesis and enhancing performance parameters.

As defined herein, “hematological disorder” includes abnormalities related to the blood and blood forming tissues. Hematological disorders may be caused by many factors, including, but not limited to, disease, drugs, radiation, genetics, or environmental factors.

As defined herein, “erythropoiesis” includes the formation of red blood cells.

As defined herein, “performance parameters” include physical and mental activities that depend on the oxygen carrying capacity of the blood.

As defined herein, “animal” includes any living organism having a circulatory system comprising, among other components, hemoglobin and red blood cells for the transportation of oxygen throughout the body of the animal.

Although Echinacea is known to stimulate the immune system, it has not been previously disclosed that Echinacea provides a hematinic effect. The present invention is based on the unexpected discovery that Echinacea can be used as an effective and nontoxic agent for the prevention of hematological disorders, for the treatment of hematological disorders, for stimulating erythropoiesis and for enhancing performance parameters in an animal, including a human.

The Echinacea for use in the present invention is preferably from a medicinally active species of Echinacea, more preferably from Echinacea pallida and Echinacea purpurea and most preferably from Echinacea augustifolia, or a combination thereof. The entire Echinacea plant or a part of the Echinacea plant including the stems, leaves, flowers, root and any combination thereof can be used to provide the Echinacea for use in the present invention. Alternatively, an extract of the Echinacea plant or of a part of the Echinacea plant including the stems, leaves, flowers, root and any combination thereof can be used in the present invention.

Various methods for extracting Echinacea are known to those skilled in the art. These include, but are not limited to, methanol, methanol and water, and supercritical fluid extraction of the Echinacea plant or a part thereof. Further, the extract can be dried to form a powder, or can be fractionated, purified and dried to form a powder. Alternatively, an extract of the Echinacea plant, or of a part thereof, or of a fractionated and purified extract of the Echinacea plant, or of a part thereof, can be purchased from commercial sources including, but not limited to, Indena Spa (Milano, Italy).

Echinacea plants and parts thereof, or extracts of Echinacea plants and parts thereof, are administered to an animal, including a human, in a pharmaceutically acceptable carrier. Advantageously, the Echinacea composition can be prepared by uniformly and intimately bringing into association the Echinacea plant or a part thereof, or an extract of an Echinacea plant or a part thereof, with a liquid carrier, with a solid carrier or with both. Liquid carriers include, but are not limited to, aqueous carriers, non-aqueous carriers or both. Solid carriers include, but are not limited to, biological carriers, chemical carriers or both. However, it is to be understood that any of the pharmaceutical carriers known to those skilled in the art to be acceptable for administration to an animal or a human can be used in the present invention.

Aqueous carriers include, but are not limited to, water, saline, physiological buffers, sucrose, ribose, glycerin, alcohol and juice. Nonaqueous carriers include, but are not limited to, oil emulsions, water-in-oil emulsions and water-in-oil-in-water emulsions. Echinacea and a pharmaceutically acceptable carrier can be prepared as liquid compositions including, but not limited to, solutions, suspensions, tinctures, and syrups,

Solid carriers include, but are not limited to, powders, tablets, capsules, particles, microparticles, microspheres, nanoparticles, and various natural or synthetic polymers that allow for sustained release of the Echinacea. Echinacea and a pharmaceutically acceptable carrier can be prepared as solid compositions including, but not limited to, powders, tablets and capsules.

Further, the Echinacea composition of the present invention can be used with any one, all, or any combination of agents regardless of the carrier used to present the Echinacea to the animal. These include, but are not limited to, vitamins, minerals, neutraceuticals, free radical scavengers, amino acids, antiseptics, antibacterials, antifungals, antivirals and immunostimulants, antioxidants, mycobacterial cell wall extract, mycobacterial cell complex, and hyaluronic acid. Mycobacterial cell wall extract, mycobacterial cell complex, and hyaluronic acid are all available from Bioniche Life Sciences, London, Ontario, Canada. The agent and the amount of the agent to be included in the Echinacea composition are well within the determination of those skilled in the art.

Vitamins are trace organic substances that are required in the diet and include co-factors and coenzymes. These include, but are not limited to, vitamins A, C, E and B12, thiamin, riboflavin, nicotinic acid, pantothenic acid, pyridoxine, biotin, folic acid, ribose, omega-3-fatty acids, omega-6-fatty acids, lipoic acid, ascorbic acid, thiamine pyrophosphates, nucleotides, flavins, folic acid, choline, carotenes and carnitine, coenzyme Q-10, L-tryptophan, glutathione, L-carnitine, methylsulfonylmethane (MSM), L-glutamine, N-acetyl-d-glucosamine.

Minerals are trace organic substances that are required in the diet. These include, but are not limited to, calcium, chromium, copper, iodine, iron, magnesium, manganese, phosphorous and selenium.

Neutraceuticals are botanicals or herbs that are used as dietary supplements. These include, but are not limited to Elecampane—Helenii rhizoma, Hawthorne—Crataegus spp, Golden Seal—Hydrastis canadensis, Northern Prickly Ash—Zanthoxylum americanum, Willow—Salix alba, Mullein—Verbascuin thapsus, Milk Thistle—Silybum marianum, Ginkgo—Ginkgo biloba, St. Johns wort—Hypericum perforatum, Eleuthero—Eleutherococcus senticosus, Eyebright—Euphrasia Americana, Cats Claw—Uncaria tomentos, Garlic—Allium sativum, Oregon Grape Root—Berberis aquifolium, Paud″Ardco—Tabebuia spp, American Ginseng—Panax ginseng, Korean Ginseng—Panax quinquefolius, Burdock Root—Arctium lappa, Celery Seed—Apium graveolens, Dandelion—Taraxacum officinale, Mint—Mentha piperita, Mint—Mentha spicata, Horse Mint (Bergamont)—Monarda spp, Stinging Nettles—Urtica dioica, Rose Hips—Rosa rugosa, Rose Hips—Rosa canina, Thyme—Thymus vulgaris, Fenugreek—Trigonella foenum-gracecum, Red Clover—Trifolium pratense, Kelp seaweed—Fucus vesiculosus, Raspberry—Rubus idaeus, Calendula—Calendula officinalis, Horse Chestnut—Aesculus hippocastanum, Bilberry/Blueberry—Vaccinium myrtiillus/angustifolia, Evening Primrose—Oenothera biennis, Flax Seed—Linum usitatissumum, Black Current—Ribes nigrum, Borrage—Borago officinalis, Black Cohosh—Cimicifuga racemosa, Cranberry—Vaccinium macrocarpon, Chamomile—Matricaria chamomilla, Comfry—Symphytum officinale, Coltsfoot—Tussilago farfara, Horseradish—Cochlearia armoracia, Devils Claw—Harpogophytum procumbens, Golden Rod—Solidago virgaurea, Hops—Humulus lupulus, Valerian—Valeriana officinalis, Yarrow—Achillea millefoliun, Slippery Elm—Ulmus rubra, Plantain—Plantago lanceolata, Lobelia—Lobelia inflata, Aloe—Aloe vera, Mellissa—Melissa officinalls, Mayapple—Podophyllum peltatum, Balm of Gilead Bud—Populi gemma, and Elderberry—Sainbuci flos.

The Echinacea composition of the present invention may also include adjuvants and excipients that provide bulk and bindability, that provide stabilization and that enhance the appearance or flavor of the Echinacea composition. These are known to those skilled in the art and include, but are not limited to binders, flow enhancers, disintegrants, granulating agents, coating agents, flavoring agents, and coloring agents. These are present in an amount preferably from about 0% to about 99% by weight, more preferably from about 1% to about 90% by weight, and most preferably from about 4% to about 80% by weight.

In an aspect of the invention, powdered extract of Echinacea augustifolia root is suspended and stabilized in a fruit juice. In another aspect, powdered extract of Echinacea augustifolia root is combined with cellulose to form a tablet using tabletting methods known to those skilled in the art. In another aspect, powdered extract of Echinacea augustifolia root is inserted into a gelatin capsule using encapsulating methods known to those skilled in the art.

The Echinacea composition of the present invention is administered to animals, including humans, in a dose effective to induce a hematinic effect. The dose administered, the number of doses administered and the dose schedule will depend on the condition being treated, the severity of the condition being treated and other clinical factors such as the size, weight and condition of the recipient and the route of administration. The dose of Echinacea to be administered and the schedule of administration can be determined by the practitioner using standard techniques. In addition, in vitro assays for red blood cells and red blood cell components may optionally be employed to help identify optimal ranges for Echinacea administration.

Preferably, the Echinacea composition is administered orally wherein the Echinacea comprises an amount between about 10 mg and 20,000 mg, more preferably between about 25 mg and about 12,000 mg and most preferably-between about 100 mg and about 8000 mg per dose. For administration as a liquid, the volume of the Echinacea composition per dose is preferably between about 1 ml and about 400 ml, more preferably between about 5 ml and about 200 ml, and most preferably between about 10 ml and about 100 ml, and the percent of Echinacea per dose is preferably from about 0.01% to 50% by weight, more preferably from about 0.1% to about 25% by weight and most preferably from about 1% to about 10% by weight.

Because of its hematinic effects, the Echinacea composition of the present invention is effective for preventing a hematological disorder, for treating a hematological disorder, for stimulating erythropoiesis and for enhancing performance parameters that depend on the oxygen carrying capacity of the blood.

Hematological disorders are associated with conditions that can be prevented and treated by the Echinacea composition of the present invention, and include, but are not limited to, anemia of premature birth, anemia of the newborn, megaloblastic anemia, anaplastic anemia, hypoplastic anemia, parasitic anemia, drug-induced anemia, erythroblastopenia, anemia of renal failure, anemia of hypothyroidism, anemia of infection and inflammation, anemia of marrow replacement, primary refractory anemia, hemolytic anemia, sickle cell anemia, anemia associated with HIV infection, hemolgobinopathies, thalassemia, immune hemolytic anemia polycythemia, emphysema, asthma, and hematological disorders caused by environmental or therapeutic conditions, such as, for instance, exposure to radiation, chemicals or drugs. Hematological parameters can be measured by methods known to those skilled in the art including, but not limited to, complete blood count, red blood cell count, hemoglobin concentration, hematocrit, mean corpuscular volume, mean corpuscular hemoglobin, mean corpuscular hemoglobin concentration, arterial oxygen tension and venous oxygen tension.

Performance parameters that can be increased by the Echinacea composition of the present invention include, but are not limited to, stamina, endurance, strength, agility, mental acuity, mental alertness and stress reduction. Performance parameters can be measured by methods known to those skilled in the art including, but not limited to, treadmill tests that assess the point of fatigue, measurement of athletic performance such as running speed, determination of increased strength with weight resistance, and measurement of the ability to perform tasks at decreased oxygen tensions.

In addition, performance can be measured by tests known to those skilled in the art including, but not limited to: treadmill tests for the point of voluntary exhaustion; physiological tests of biological variables including, but not limited to, heart rate, heart rate recovery, cardiac recovery index, respiratory rate, thermoregulation, oxygen tension and lactic acid production; and, psychological tests including, but not limited to, tests for mental agility, mental alertness and stress levels.

The following examples will serve to further illustrate the present invention without, at the same time, however, constituting any limitation thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

For Examples 1-5, Equine Serum Profiles (ESP) were performed on the Hitachi 911 Biochemical analyzer by VitaTech Veterinary Laboratory Services of Markham Ontario. The serum profile included measurement of an albumin/globulin ratio, albumin, alkaline phosphatase, calcium, chloride, cholesterol, creatine phosphokinase, creatinine, glucose, phosphorus, potassium, total protein, aspartate aminotransferase, sodium and sodium/potassium ratio. An ESP was performed on the blood samples drawn once each week during each sampling period. Testing did not indicate any abnormal results, nor did the test subjects appear to experience any side effects from the study.

### EXAMPLE 1

Echinacea Syrup Composition

In an aspect of the present invention, a powdered extract of Echinacea augustifolia root, standardized to 4% echinacocide, was obtained from Indena Spa (Milano, Italy). Echinacea syrup was prepared using the ingredients listed in Table 1.

TABLE 1
Echinacea augustifolia syrup composition

| Ingredients | Amount (g) | Weight Percent |
|---|---|---|
| Echinacea augustifolia | 220 g | 4.2% |
| (1:4 extract of root) | | (5.5% by volume) |
| Sucrose (USP) | 2000 g | 38.2% |
| Water (USP) | 3000 g | 57.4% |
| Sodium benzoate | 5 g | 0.1% |
| Lecithin | 5 g | 0.1% |
| Total | 5,230 g | 100% |

The sucrose, water, sodium benzoate and lecithin were mixed together and the powdered Echinacea extract was suspended in the mixture to form Echinacea syrup. Each 20 ml of the Echinacea syrup contained 1.1 g of a 1:4 extract of Echinacea root.

### EXAMPLE 2

Echinacea Tablet Composition

Powdered extract of Echinacea augustifolia root, standardized to 4% echinacocide, was obtained from Indena Spa (Milano, Italy). An Echinacea tablet was prepared using the ingredients listed in Table 2.

TABLE 2
Echinacea augustifolia capsule composition

| Ingredients | Amount (g) | Dry Weight Percent |
|---|---|---|
| Echinacea augustifolia | 52 g | 26% |
| (1:4 extract of root) | | |
| Microcrystalline cellulose | 30 g | 15% |
| Sodium | 10 g | 5% |
| carboxymethylcellulose | | |
| Hydroxypropylcellulose | 10 g | 5% |
| Stearic acid | 50 g | 25% |
| Silicon dioxide | 24 g | 12% |
| Starch | 24 g | 12% |
| Total | 200 g | 100% |

The microcrystalline cellulose, sodium carboxymethylcellulose, hydroxy-propylcellulose, stearic acid and silicon dioxide are mixed together by methods known to those skilled in the art, the powdered Echinacea is added and 500 mgs of the Echinacea composition are packed into a standard gelatin capsule. Each 500 mg capsule contains 130 mg of a 1:4 extract of Echinacea root.

### EXAMPLE 3

Horses

Eight healthy horses, 7 mares and 1 gelding, were used in this study. Two of the horses were thoroughbreds, 3 were standardbreds, 2 were Canadians and 1 was a quarter horse. The horses ranged in age from 2 to 9 years of age. The horses were bedded on straw and fed a hay/concentrate diet that met their nutritional requirements. During the day, weather permitting, they were turned out to pasture and during the night they were stabled. They were acclimatized to their feed and accommodation for a minimum of 2 weeks prior to commencement of the study.

Each horse was randomly allocated a number corresponding to a jug containing either 30 cc of placebo syrup (Example 1 without Echinacea) or 20 cc of Echinacea syrup (Example 1) diluted to 30 cc with 10 cc of placebo syrup. Each horse received its assigned supplement twice daily as a top dress on its feed for 42 days (supplementation period I).

The 42-day supplementation period was followed by a 14-day washout period, during which the horses received no supplementation. After the 14-day washout period, each horse previously in the Echinacea syrup group received 30 cc of placebo syrup and each horse previously in the placebo syrup group received Echinacea syrup twice daily as a top dress on its feed for 42 days (supplementation period II).

In these experiments, each horse served as its own control. In total, eight horses were supplemented with Echinacea syrup for 42 days and eight horses were supplemented with placebo syrup for 42 days.

### EXAMPLE 4

Blood Profiles

All blood profiles were conducted at VitaTech Veterinary Laboratory Services (Markham, Ontario). Complete blood counts were performed using a Technicon H* 1 (Bayer, Inc., Etobicoke, Ontario, CA). Complete blood counts included red blood cell count, hemoglobin concentration, nucleated red blood cell count, mean corpuscular volume, mean corpuscular hemoglobin and mean corpuscular hemoglobin concentration.

Paired difference t-tests were performed using the means procedure in SAS (SAS, Inc. 1990, Cary, N.C., USA). Differences between control and treatment were calculated for each horse at each sample time. The means procedure was used to test whether differences were significantly different than zero. Differences between sample time 0 and each of the experimental sample times were used to test whether differences over time were significantly different from zero. Differences were determined to be significant if p<0.05.

### EXAMPLE 5

Results

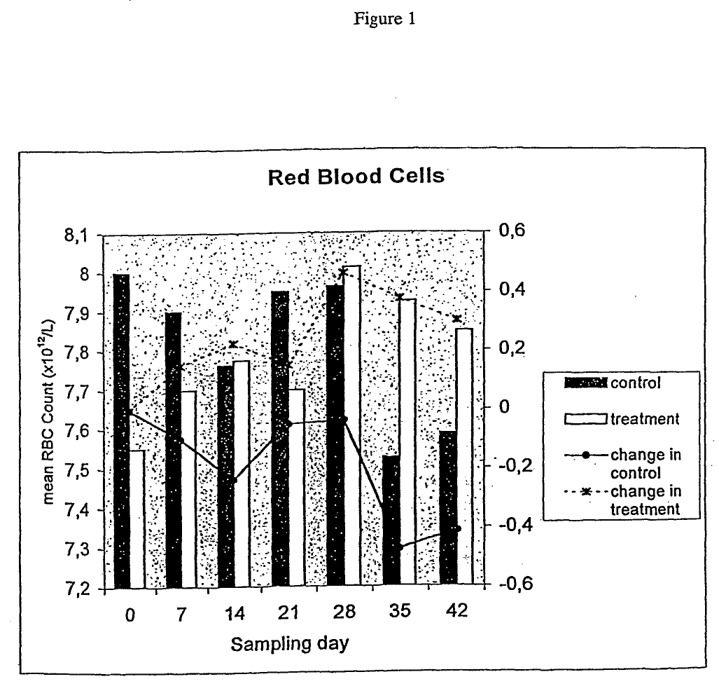

As shown in Table 3 and in FIG. 1, there was a significant increase over time in mean red blood cell counts of horses receiving Echinacea syrup as a dietary supplement compared to horses receiving placebo syrup as a dietary supplement.

TABLE 3
Red blood cell (RBC) counts

| | | | Difference | | |
|---|---|---|---|---|---|
| | | | Between | Standard | |
| Sampling | Means | | Control and | Error or | p-Value of |
| Day | Control | Treatment | Treatment | Difference | Difference |
| 0 | 8.000 | 7.5500 | 0.4500 | 2.2400221 | 0.0601 |
| 7 | 7.900 | 7.7000 | 0.2000 | 1.1686974 | 0.2808 |
| 14 | 7.7625 | 7.7750 | −0.0125 | −0.0626575 | 0.9518 |
| 21 | 7.9500 | 7.7000 | 0.2500 | 2.7583864 | 0.0282 |
| 28 | 7.9625 | 8.0125 | −0.0500 | −0.2522625 | 0.8081 |
| 35 | 7.5250 | 7.9250 | 0.2500 | 1.5275252 | 0.1405 |
| 42 | 7.5875 | 7.8500 | −0.2625 | −1.7793689 | 0.1184 |

Significant difference between treatment and control over time p = 0.0082

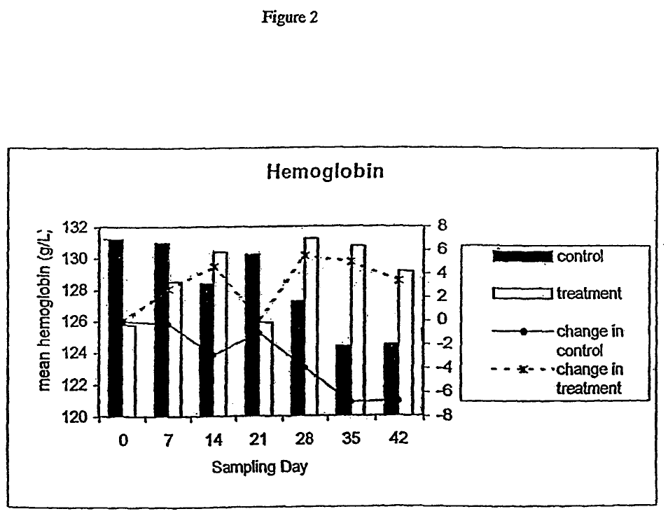

As shown in Table 4 and in FIG. 2, there was a significant increase over time in mean hemoglobin concentration of horses receiving Echinacea syrup as a dietary supplement as compared to horses receiving placebo syrup as a dietary supplement.

TABLE 4
Hemoglobin concentrations

| | | | Difference | | |
|---|---|---|---|---|---|
| | | | Between | Standard | |
| Sampling | Means | | Control and | Error or | p-Value of |
| Day | Control | Treatment | Treatment | Difference | Difference |
| 0 | 131.2500 | 125.7500 | 5.5000 | 2.9880715 | 0.1082 |
| 7 | 131.0000 | 128.5000 | 2.5000 | 2.8784917 | 0.4139 |
| 14 | 128.3750 | 130.3750 | -2.0000 | 3.3380918 | 0.5680 |
| 21 | 130.2500 | 125.8750 | 4.3750 | 1.4134242 | 0.0174 |
| 28 | 127.2500 | 131.2500 | -4.0000 | 4.1576092 | 0.3681 |
| 35 | 124.3750 | 130.7500 | -6.375 | 3.3000947 | 0.0947 |
| 42 | 124.5000 | 128.1250 | -4.625 | 2.1871173 | 0.0723 |

Significant difference between treatment and control over time p = 0.0216

There was a significant difference in complete blood count (CBC) parameters between treatment and control groups. As shown in Table 5, there was a significant increase over time in mean red blood cell counts and in mean hemoglobin concentrations of the horses receiving Echinacea syrup as a dietary supplement as compared to horses receiving placebo syrup as a dietary supplement.

TABLE 5
Differences in CBC parameters between Echinacea supplemented and
placebo supplemented groups

| Blood Parameters | Sampling Day | Mean Difference | p-value |
|---|---|---|---|
| Haematocrit | 0 | 2.13 | 0.03 |
| | 35 | −2.38 | 0.03 |
| | 42 | −1.38 | 0.04 |
| Haemoglobin | 21 | 4.38 | 0.02 |
| | 7 | −0.63 | 0.01 |
| Mean Corpuscular Volume | 7 | −0.63 | 0.01 |
| (MCV) | | | |
| | 35 | −0.88 | 0.006 |
| Red Blood Cell Count | 21 | 0.25 | 0.03 |
| (RBC) | | | |
| | 35 | −0.40 | 0.05 |
| Lymphocytes (actual) | 35 | −0.54 | 0.006 |
| Neutrophils (actual) | 35 | 0.80 | 0.05 |

Significant difference over time p < 0.01

Significant differences in CBC parameters resulted from supplementation with Echinacea syrup, whereas no significant differences resulted from supplementation with placebo syrup. As shown in Table 6, mean red blood cell counts and mean hemoglobin concentrations of horses on supplementation with Echinacea syrup were significantly greater than baseline values (time 0), whereas mean red blood cell counts and mean hemoglobin concentrations of horses on supplementation with placebo syrup were not significantly different from baseline values (time 0).

TABLE 6
Changes in CBC Parameters Different from “0”

| Blood | Value at Day 0 | Mean | | |
|---|---|---|---|---|
| Parameter | minus Day: | Difference | Control | Treatment |
| Haematocrit | 35 | 2.25 | 0.02↓ | |
| | 28 | −2.63 | | 0.03↑ |
| | 35 | −2.25 | | 0.03↑ |
| Haemoglobin | 28 | 4.00 | 0.03↓ | |
| | 42 | 6.75 | 0.02↓ | |
| MCH | 28 | 0.50 | | 0.03↓ |
| MCHC | 21 | 8.75 | | 0.005↓ |
| | 28 | 10.38 | | 0.0008↓ |
| | 35 | 7.75 | | 0.04↓ |
| | 42 | 8.63 | | 0.003↓ |
| MCV | 7 | −0.63 | | 0.05↑ |
| | 42 | −0.63 | | 0.01↑ |
| RBC | 35 | 0.48 | 0.04↓ | |
| | 42 | 0.41 | 0.04↓ | |
| Neutrophils | 35 | 0.98 | | 0.04↓ |
| (actual) | | | | |

Significant difference from time 0 p < 0.05

In conclusion, animals receiving Echinacea syrup supplementation showed significant increases in mean red blood cell counts and in mean hemoglobin concentrations. The Echinacea supplement of the present invention is a hematinic agent that provides a positive benefit to the health and performance capacity of the treated animals.

### EXAMPLE 6

Complete Blood Counts

Twelve normal human volunteers are randomly allocated to Group A or to Group B. Group A volunteers receive 30 cc of orange juice containing of powdered Echinacea augustifolia root, standardized to 4% echinacocide, twice per day for 30 days as a dietary supplement. Group B volunteers receive 30 cc of orange juice (placebo) twice per day for 30 days as a dietary supplement. Blood samples are collected from each volunteer at day 0 (control) and at days 5, 10, 15, 20, 25 and 30 after initiation of dietary supplementation. Complete blood counts are performed on each blood sample. There is a significant increase over time in red blood cell counts and hemoglobin concentrations in volunteers receiving orange juice and Echinacea as a dietary supplement compared to volunteers receiving only orange juice as a dietary supplement.

### EXAMPLE 7

Performance Parameters

Ten racehorses are randomly allocated to Group A or to Group B. Group A horses receive the Echinacea syrup of Table 1 as a dietary supplement twice per day for 60 days. Group B horses receive the placebo syrup of Table 1 without the Echinacea (placebo) as a dietary supplement twice per day for 60 days. After the 60 day time period the performance of Group A and Group B horses is assessed for improved race time and increased endurance. Group A horses show improvement in race time and an increase in endurance after 60 days of Echinacea syrup supplementation. Group B horses show no improvement in race time and no increase in endurance after 60 days of placebo syrup supplementation.

### EXAMPLE 8

Hematological Disorder

Three patients with cancer are treated with chemotherapeutic agents that result in the induction of anemia. Each of the patients is given Echinacea tablets as a dietary supplement 2 times per day for 4 weeks. Blood is drawn weekly and complete blood counts are performed on each blood sample. At the end of the 4 weeks, there is a significant increase in the red blood cell count and hemoglobin concentration in each of the patients.

### EXAMPLE 9

Ten dogs are randomly allocated to Group A or to Group B. Group A dogs receive the Echinacea syrup of Table 1 as a dietary supplement twice per day for 60 days. Group B dogs receive the placebo syrup of Table 1 without the Echinacea (placebo) as a dietary supplement twice per day for 60 days. After the 60 day time period the performance of Group A and Group B dogs is assessed for improved race time and increased endurance. Group A dogs show improvement in race time and an increase in endurance after 60 days of Echinacea syrup supplementation. Group B dogs show no improvement in race time and no increase in endurance after 60 days of placebo syrup supplementation.

### EXAMPLE 10

Ten calves are randomly allocated to Group A or to Group B. Group A calves receive the Echinacea syrup of Table 1 as a dietary supplement twice per day for 60 days. Group B calves receive the placebo syrup of Table 1 without the Echinacea (placebo) as a dietary supplement twice per day for 60 days. After the 60 day time period, the hematologic factors indicating hematinic effect of Group A and Group B calves are assessed for increased red blood cells, increased hemoglobin, and increased hematocrit. Group A calves show an increase in hematologic factors after 60 days of Echinacea syrup supplementation. Group B calves show no increase in hematologic factors after 60 days of placebo syrup supplementation.

### EXAMPLE 11

Ten swine are randomly allocated to Group A or to Group B. Group A swine receive the Echinacea syrup of Table 1 as a dietary supplement twice per day for 60 days. Group B swine receive the placebo syrup of Table 1 without the Echinacea (placebo) as a dietary supplement twice per day for 60 days. After the 60 day time period the hematologic factors of red blood cell count, hemoglobin and hematocrit of Group A and Group B swine are assessed for an increase indicating a hematinic effect. Group A swine show an increase in hematologic factors after 60 days of Echinacea syrup supplementation. Group B swine show no increase in hematologic factors after 60 days of placebo syrup supplementation.

It should be understood, of course, that the foregoing relates only to a preferred embodiment of the present invention and that numerous modifications or alterations may be made therein without departing from the spirit and the scope of the invention as set forth in the appended claims.

We claim:

1. A method of causing a hematinic effect in an animal or a human, comprising administering a composition comprising Echinacea and a pharmaceutically acceptable carrier in an amount effective to cause a hematinic effect.

2. The method of claim 1, wherein the hematinic effect is selected from preventing a hematological disorder, treating a hematological disorder, stimulating erythropoiesis, enhancing performance parameters, or a combination thereof.

3. The method of claim 2, wherein the stimulation of erythropoiesis is selected from increasing the mean number of red blood cells or increasing the mean hemoglobin concentration.

4. The method of claim 2, wherein the hematologic disorder is associated with anemia of premature birth, anemia of the newborn, megaloblastic anemia, aplastic anemia, hypoplastic anemia, parasitic anemia, anemia of hypothyroidism, anemia of infection, hemolytic anemia, sickle cell anemia, anemia associated with HIV, erythroblastopenia, hemoglobinopathy, thalessemia, polycythemia, hemolytic anemia, anemia of renal failure, emphysema, asthma, environmental conditions, therapeutic conditions, drug administration, or any combination thereof.

5. The method of claim 1 wherein the hematinic effect is an increase in red blood cells, hemoglobin concentration, hematocrit, mean corpuscular hemoglobin, mean corpuscular volume, mean corpuscular hemoglobin concentration, arterial oxygen tension, venous oxygen tension, or any combination thereof.

6. The method of any of claims 1, 2, 3, 4, or 5, wherein an Echinacea species is Echinacea pallida, Echinacea purpurea, Echinacea augustifolia, or any combination thereof.

7. The method of any of claims 1, 2, 3, 4, or 5, wherein the composition is administered at least once daily.

8. The method of any of claims 1, 2, 3, 4, or 5, wherein the amount of Echinacea in the composition is from about 10 mg to about 6000 mg, from about 25 mg to about 4,000 mg, or from about 50 mg to about 3,000 mg per dose.

9. The method of claim 1, wherein the Echinacea is prepared from any part of the Echinacea plant.

10. The method of claim 1, wherein the Echinacea is prepared from an extract of any part of the Echinacea plant.

11. The method of claim 1, wherein the composition is administered orally.

12. The method of claim 11, wherein the composition is administered in a tablet, a gel capsule, a liquid, or any combination thereof.

13. The method of claim 12, wherein the liquid is administered in a volume of from about 1 ml to about 400 ml, from about 5 ml to about 200 ml, or from about 10 ml to about 100 ml.

14. The method of claim 12, wherein the percent of Echinacea per dose is from about 0.01% to about 50%, from about 0.1% to about 25%, or from about 1% to about 10%.

15. The method of claim 1, further comprising administration of at least one agent selected from vitamins, minerals, neutraceuticals, free radical scavengers, amino acids, antiseptics, antibacterials, antifungals, antivirals, immunostimulants, antioxidants, mycobacterials cell wall extracts, mycobacterial cell complexes, hyaluronic acids, adjuvants, excipients, or any combination thereof.

16. The method of claim 15 wherein the at least one agent is present in a concentration of from about 0% to about 99%, from about 1% to about 90%, or from about 4% to about 80%.

17. Use of a composition comprising Echinacea and a pharmaceutically acceptable carrier to produce a hematinic effect in an animal or a human comprising administration to the animal or the human of an effective amount of the composition, wherein the effective amount produces a hematinic effect in the animal or the human.
